(19) United States
(12) Patent Application Publication
(10) Pub. No.: US 2025/0277251 A1
(43) Pub. Date: Sep. 4, 2025

(54) DETECTION DEVICE

(71) Applicant: Japan Display Inc., Tokyo (JP)

(72) Inventors: Akihiko FUJISAWA, Tokyo (JP); Tomoya TEZEN, Tokyo (JP); Kaoru ITO, Tokyo (JP); Daichi ABE, Tokyo (JP)

(21) Appl. No.: 19/062,558

(22) Filed: Feb. 25, 2025

(30) Foreign Application Priority Data
Mar. 1, 2024 (JP) 2024-031590

(51) Int. Cl. C12Q 1/16; G01N 33/483; H10F 39/00

(52) U.S. Cl. CPC C12Q 1/16; G01N 33/4833; H10F 39/804; H10F 39/8053

(57) ABSTRACT

According to an aspect, a detection device includes: an optical sensor comprising photodetection elements arranged in a planar configuration; a light source configured to emit light to the photodetection elements; and an object placement member. The light source, the object placement member, and the optical sensor are arranged in the order as listed. The optical sensor is configured to obtain baseline data in an initial state after power-on. The detection device is configured to: obtain difference data based on a difference between the baseline data and sensor values obtained from the photodetection elements at intervals of a predetermined period of time; and output, when the difference data has exceeded a first threshold, either an alert indicating detection of the objects to be detected in an initial moisture region indicating presence of moisture in the initial state, or an alert indicating detection of the objects to be detected.

### CROSS-REFERENCE TO RELATED APPLICATION

This application claims the benefit of priority from Japanese Patent Application No. 2024-031590 filed on Mar. 1, 2024, the entire contents of which are incorporated herein by reference.

### BACKGROUND

### 1. Technical Field

What is disclosed herein relates to a detection device.

### 2. Description of the Related Art

Japanese Patent Application Laid-open Publication No. 2018-033430 discloses an image acquisition device that includes an optical sensor, a container to contain microorganisms and a culture medium, and a light source, and acquires, over time, images indicating a growth of the microorganisms in the container.

In such a detection device, if moisture (e.g., beads of water) adheres to the container, an object to be detected as a detection target may be difficult to be detected well.

For the foregoing reasons, there is a need for a detection device capable of improving the accuracy of the detection.

### SUMMARY

According to an aspect, a detection device includes: an optical sensor comprising a plurality of photodetection elements arranged in a planar configuration; a light source configured to emit light to the photodetection elements; and an object placement member that has a light-transmitting property and on which a plurality of objects to be detected are to be placed. The light source, the object placement member, and the optical sensor are arranged in the order as listed. The optical sensor is configured to obtain baseline data in an initial state after power-on. The detection device is configured to: obtain difference data based on a difference between the baseline data and sensor values obtained from the photodetection elements at intervals of a predetermined period of time; and output, when the difference data has exceeded a first threshold, either an alert indicating detection of the objects to be detected in an initial moisture region indicating presence of moisture in the initial state, or an alert indicating detection of the objects to be detected.

### BRIEF DESCRIPTION OF THE DRAWINGS

FIG. 1 is a sectional view schematically illustrating a detection device according to an embodiment;

FIG. 2 is a block diagram illustrating a configuration example of the detection device according to the embodiment;

FIG. 3 is a circuit diagram illustrating a sensor pixel;

FIG. 4 is a block diagram illustrating a configuration example of a detection circuit according to the embodiment;

FIG. 5 is a block diagram illustrating a configuration example of a sensor control circuit according to the embodiment;

FIG. 6 is a schematic diagram schematically illustrating states of growth of objects to be detected;

FIG. 7 is a graph schematically illustrating a relation between a sensor value and time in detection of the objects to be detected;

FIG. 8 is a graph schematically illustrating a relation between difference data and time in the detection of the objects to be detected;

FIG. 9 is an explanatory chart for explaining the sensor value, the difference data, and the detected objects in the detection of the objects to be detected;

FIG. 10 is a schematic diagram schematically illustrating states of evaporation of moisture;

FIG. 11 is a graph schematically illustrating a relation between the sensor value and time in detection of the moisture;

FIG. 12 is a graph schematically illustrating a relation between the difference data and time in the detection of the moisture;

FIG. 13 is an explanatory chart for explaining the sensor values, the difference data, and a detected initial moisture region in the detection of the moisture;

FIG. 14 is a graph schematically illustrating a relation between the sensor value and time in the detection of the moisture according to a modification of the embodiment;

FIG. 15 is a graph schematically illustrating a relation between the difference data and time in the detection of the moisture according to the modification;

FIG. 16 is an explanatory chart for explaining the sensor values, the difference data, and the detected initial moisture region in the detection of the moisture according to the modification;

FIG. 17 is a graph schematically illustrating a relation between the sensor value and time in the detection of the objects to be detected and the moisture;

FIG. 18 is a graph schematically illustrating a relation between the difference data and time in the detection of the objects to be detected and the moisture;

FIG. 19 is a schematic diagram schematically illustrating the states of the growth of the objects to be detected and the states of the evaporation of the moisture;

FIG. 20 is an explanatory chart for explaining the sensor values, the difference data, and the objects to be detected and the initial moisture region that have been detected in the detection of the objects to be detected and the moisture;

FIG. 21 is a flowchart for explaining a method for obtaining baseline data;

FIG. 22 is a flowchart for explaining a method for obtaining the difference data;

FIG. 23 is a flowchart for explaining a method for detecting the objects to be detected and the output of alerts; and

FIG. 24 is a flowchart for explaining a method for detecting the moisture.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present disclosure, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

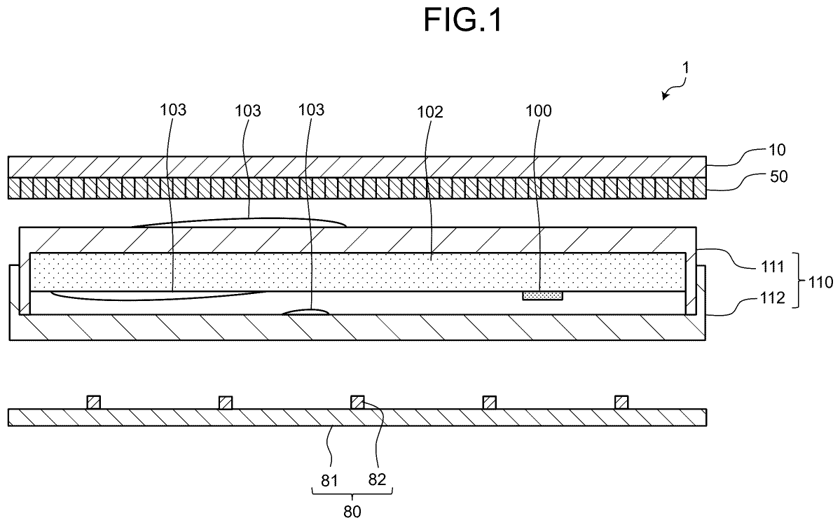

FIG. 1 is a sectional view schematically illustrating a detection device according to an embodiment. As illustrated in FIG. 1, a detection device 1 includes an optical sensor 10, an optical filter layer 50, a container 110 for accommodating an object to be detected 100, and a light source 80. In the detection device 1, the light source 80, the container 110 (object to be detected 100), the optical filter layer 50, and the optical sensor 10 are arranged in this order.

The object to be detected 100 is, for example, micro-objects such as bacteria. The bacteria or the like that have been cultured on a culture medium 102 (e.g., agar) and grown into a clump large enough to be visible may be referred to as a colony. The detection device 1 is a biosensor that detects the micro-objects such as the bacteria. The object to be detected 100 is not limited to the bacteria and may be other micro-objects such as cells.

The container 110 includes a container body 111 and a cover member 112. The container 110 is a Petri dish, for example. The container 110 is light-transmitting. The container body 111 contains the culture medium 102, and the object to be detected 100 is cultured on the culture medium 102. That is, the container 110 (at least the container body 111, of the container body 111 and the cover member 112) is an object placement member that has a light-transmitting property and in which (on which) a plurality of the objects to be detected 100 can be placed.

In the present embodiment, the container 110 is placed upside down with respect to a normal container. That is, the normal container is placed such that the container body is located on the lower side and the cover member is located on the upper side. In contrast, the container 110 according to the present embodiment is placed such that the container body 111 is located on the upper side and the cover member 112 is located on the lower side. Specifically, the objects to be detected 100 such as the bacteria are placed on the upper side of the culture medium 102 and cultured, and when imaging the objects to be detected 100, the container 110 is placed upside down such that the objects to be detected 100 is placed on the lower side of the culture medium 102. The objects to be detected 100 serving as detection targets and the culture medium 102 are contained in the container 110 and placed between the optical sensor 10 and the light source 80.

In the container 110, water vapor may be generated from the culture medium 102, and moisture 103 (e.g., beads of water) may adhere to the culture medium 102 or the cover member 112 of the container 110. Alternatively, the moisture 103 may adhere to the outer surface of the container 110 (upper surface of the container body 111 in FIG. 1). In this case, the intensity of light from the light source 80 changes when the light passes through the moisture 103, which may make it difficult to distinguish between the moisture 103 and the objects to be detected 100 by using sensor values So detected by the optical sensor 10. Alternatively, if the objects to be detected 100 are placed so as to overlap the moisture 103, it may be difficult to detect well the objects to be detected 100. A detailed method for detecting the objects to be detected 100 and the moisture 103 will be described later with reference to FIG. 6 and the subsequent drawings.

The optical sensor 10 is a detection device that includes a plurality of photodetection elements 30 arranged in a plane. The photodetection elements 30 are photodiodes, for example. More specifically, the photodetection elements 30 are positive-intrinsic-negative (PIN) photodiodes using inorganic semiconductors or organic photodiodes (OPDs) using organic semiconductors.

The optical filter layer 50 is a light directivity control element disposed between a plurality of light-emitting elements 82 (light source 80) and the photodetection elements 30 (optical sensor 10). More specifically, the optical filter layer 50 is provided between the photodetection elements 30 of the optical sensor 10 and the container 110. The optical filter layer 50 is disposed so as to face the photodetection elements 30 of the optical sensor 10. The optical filter layer 50 is an optical element that transmits, toward the photodetection elements 30, components of light emitted from the light-emitting elements 82 and traveling in a direction orthogonal to the optical sensor 10. The optical filter layer 50 is also called collimating apertures or a collimator.

The light source 80 includes a light source substrate 81 and the light-emitting elements 82. The light-emitting elements 82 are point light sources provided correspondingly to the photodetection elements 30 of the optical sensor 10. The light-emitting elements 82 are provided on the light source substrate 81 and arranged so as to face the photodetection elements 30 of the optical sensor 10. Each of the light-emitting elements 82 is configured as a light-emitting diode (LED), for example.

The light emitted from the light-emitting elements 82 passes through the cover member 112, the culture medium 102, the container body 111, and the optical filter layer 50, and is emitted toward the photodetection elements 30 of the optical sensor 10. The intensity of the light applied to the photodetection elements 30 differs between an area overlapping the objects to be detected 100 and an area not overlapping the objects to be detected 100. As a result, the optical sensor 10 can image the objects to be detected 100. Alternatively, when the moisture 103 is present, the intensity of the light applied to the photodetection elements 30 differs between an area overlapping the moisture 103 and an area not overlapping the moisture 103. As a result, the optical sensor 10 can detect the presence or absence of the moisture 103.

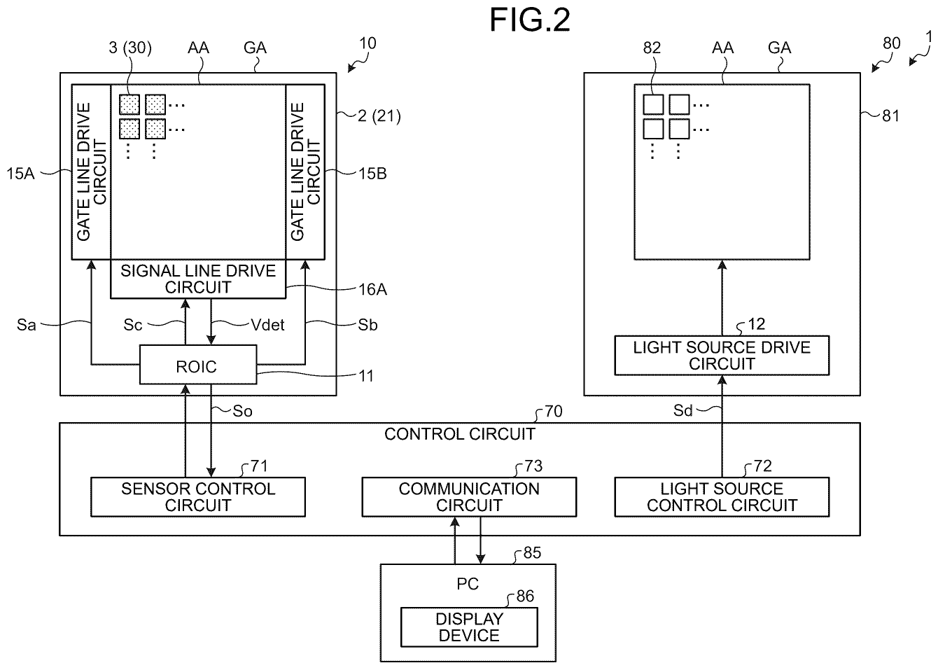

FIG. 2 is a block diagram illustrating a configuration example of the detection device according to the embodiment. As illustrated in FIG. 2, the detection device 1 further includes a control circuit 70 that controls the optical sensor 10 and the light source 80. The control circuit 70 synchronously (or non-synchronously) controls an operation of detecting the objects to be detected 100 by the optical sensor 10 and an operation of lighting the light-emitting elements 82 by the light source 80. The control circuit 70 includes, for example, a micro-controller unit (MCU), a random-access memory (RAM), an electrically erasable programmable read-only memory (EEPROM), and a read-only memory (ROM).

The optical sensor 10 includes an array substrate 2, a plurality of sensor pixels 3 (photodetection elements 30) formed on the array substrate 2, gate line drive circuits 15A and 15B, a signal line drive circuit 16A, and a detection circuit 11.

The array substrate 2 is formed using a substrate 21 as a base. Each of the sensor pixels 3 is configured with a corresponding one of the photodetection elements 30, a plurality of transistors, and various types of wiring. The array substrate 2 with the photodetection elements 30 formed thereon is a drive circuit board for driving the sensor for each predetermined detection area and is also called a backplane or an active-matrix substrate.

The substrate 21 has a detection area AA and a peripheral area GA. The sensor pixels 3 (photodetection elements 30) are arranged in a matrix having a row-column configuration in the detection area AA. The gate line drive circuits 15A and 15B, the signal line drive circuit 16A, and the detection circuit 11 are provided in the peripheral area GA.

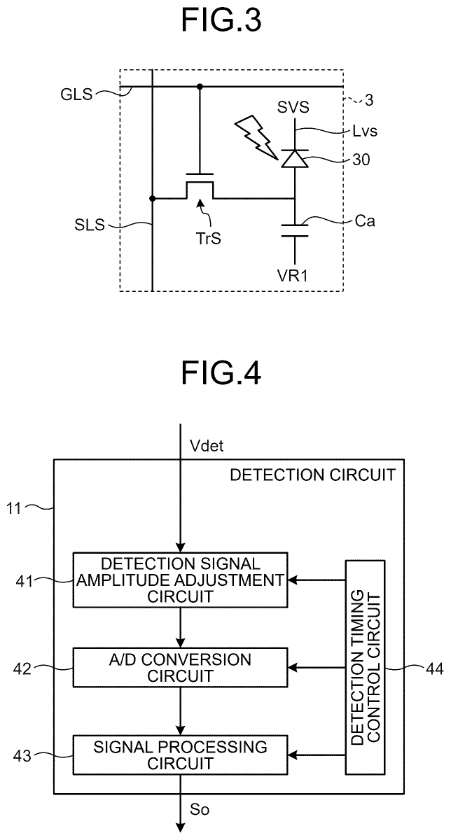

The detection circuit 11 is a circuit that supplies control signals Sa, Sb, and Sc to the gate line drive circuits 15A and 15B, and the signal line drive circuit 16A, respectively, to control operations of these circuits. Specifically, the gate line drive circuits 15A and 15B output gate drive signals to gate lines GLS (refer to FIG. 3) based on the control signals Sa and Sb. The signal line drive circuit 16A electrically couples a signal line SLS (refer to FIG. 3) selected based on the control signal Sc to the detection circuit 11. The detection circuit 11 includes a signal processing circuit that performs signal processing on a detection signal Vdet from each of the photodetection elements 30. The detection circuit 11 includes a readout integrated circuit (ROIC).

The photodetection elements 30 included in the sensor pixels 3 perform detection in response to the gate drive signals supplied from the gate line drive circuits 15A and 15B. Each of the photodetection elements 30 outputs an electrical signal corresponding to the light emitted thereto as the detection signal Vdet to the signal line drive circuit 16A. The detection circuit 11 is electrically coupled to the photodetection elements 30 via the signal line drive circuit 16A. The detection circuit 11 performs the signal processing on the detection signals Vdet from the photodetection elements 30 and outputs the sensor values So based on the detection signals Vdet to the control circuit 70. Thus, the detection device 1 detects information on the objects to be detected 100.

FIG. 3 is a circuit diagram illustrating the sensor pixel. As illustrated in FIG. 3, the sensor pixel 3 includes the photodetection element 30, a capacitive element Ca, and a transistor TrS. The transistor TrS is provided correspondingly to the photodetection element 30. The transistor TrS is configured as a thin-film transistor, and in this example, configured as an n-channel metal oxide semiconductor (MOS) thin-film transistor (TFT). The gate of the transistor TrS is coupled to the gate line GLS. The source of the transistor TrS is coupled to the signal line SLS. The drain of the transistor TrS is coupled to the anode of the photodetection element 30 and the capacitive element Ca.

The cathode of the photodetection element 30 is supplied with a power supply potential SVS from the detection circuit 11. The capacitive element Ca is supplied with a reference potential VR1 serving as an initial potential of the capacitive element Ca from the detection circuit 11.

When the sensor pixel 3 is irradiated with light, a current corresponding to the intensity of the light flows through the photodetection element 30. As a result, an electric charge is stored in the capacitive element Ca.

Turning on the transistor TrS causes a current corresponding to the electric charge stored in the capacitive element Ca to flow through the signal line SLS. The signal line SLS is coupled to the detection circuit 11 via the signal line drive circuit 16A. Thus, the optical sensor 10 of the detection device 1 can detect a signal corresponding to the intensity of the light received by the photodetection element 30 for each of the sensor pixels 3.

The transistor TrS is not limited to the n-type TFT and may be configured as a p-type TFT. The pixel circuit of the sensor pixel 3 illustrated in FIG. 3 is merely exemplary. The sensor pixel 3 may be provided with a plurality of transistors corresponding to one photodetection element 30.

FIG. 4 is a block diagram illustrating a configuration example of the detection circuit according to the embodiment. As illustrated in FIG. 4, the detection circuit 11 includes a detection signal amplitude adjustment circuit 41, an analog-to-digital (A/D) conversion circuit 42, a signal processing circuit 43, and a detection timing control circuit 44. In the detection circuit 11, the detection timing control circuit 44 performs control to synchronously operate the detection signal amplitude adjustment circuit 41, the A/D conversion circuit 42, and the signal processing circuit 43 based on a control signal supplied from the control circuit 70 (refer to FIG. 2).

The detection signal amplitude adjustment circuit 41 is a circuit that adjusts the amplitude of the detection signal Vdet output from the photodetection element 30 and is configured with an amplifier, for example. The A/D conversion circuit 42 converts analog signals output from the detection signal amplitude adjustment circuit 41 into digital signals. The signal processing circuit 43 performs signal processing on the digital signals from the A/D conversion circuit 42 and transmits the sensor values So to the control circuit 70.

Referring back to FIG. 2, the light source 80 includes a light source drive circuit 12 that drives the light- emitting elements 82 mounted on the light source substrate 81. The light-emitting elements 82 are arranged in a matrix having a row-column configuration in an area of the light source substrate 81 overlapping the detection area AA. The light source drive circuit 12 supplies a power supply voltage (an anode power supply potential and a cathode power supply potential) to the light-emitting elements 82 based on a control signal Sd from the control circuit 70 (light source control circuit 72). This operation switches the light-emitting elements 82 between on (lit state) and off (unlit state).

The number and arrangement of the light-emitting elements 82 can be changed as appropriate. The light-emitting elements 82 may emit light in a single color or may be configured to emit light having several different wavelengths. The lighting pattern of the light-emitting elements 82 can also be changed as appropriate depending on the state of the objects to be detected 100 serving as the detection target. The light-emitting elements 82 may be simultaneously turned on or may be turned on in a time-division manner on a predetermined area basis.

The control circuit 70 includes a sensor control circuit 71 that controls the optical sensor 10, the light source control circuit 72 that controls the light source 80, and a communication circuit 73. The sensor control circuit 71 and the light source control circuit 72 control the optical sensor 10 and the light source 80, respectively, so that the detection operation of the optical sensor 10 and the lighting operation of the light source 80 are synchronously performed.

The communication circuit 73 couples the control circuit 70 to an external circuit 85 in a wired or wireless manner. The external circuit 85 is a personal computer (PC), for example. The external circuit 85 is not limited thereto and may be a portable device such as a tablet computer or a smartphone. Thus, information on the objects to be detected 100 or the moisture 103 detected by the detection device 1 is output to the external circuit 85 via the communication circuit 73. By operating the external circuit 85, a user enters various conditions to be used for the detection, such as a start time and an end time of the detection of the detection device 1, and/or a first threshold Th_c and a second threshold Th_w.

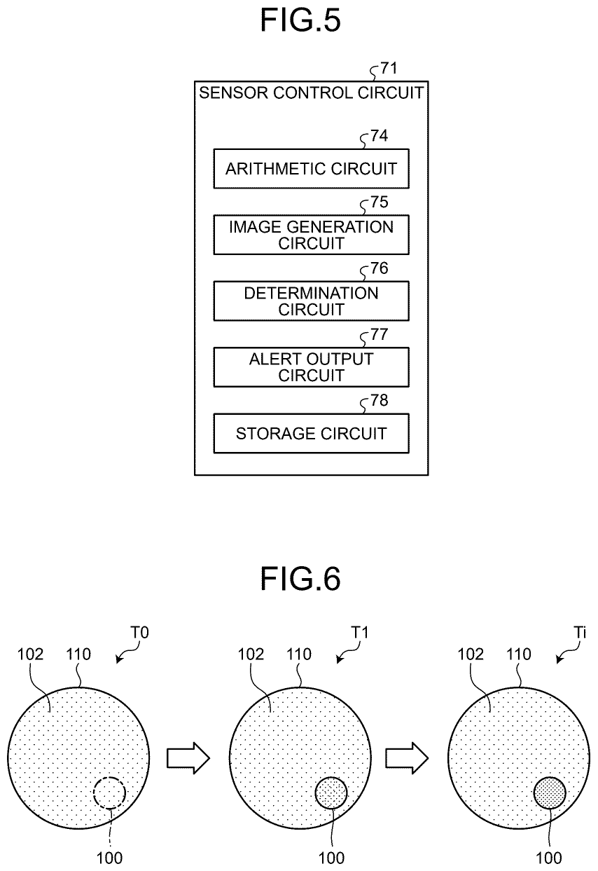

FIG. 5 is a block diagram illustrating a configuration example of the sensor control circuit according to the embodiment. As illustrated in FIG. 5, the sensor control circuit 71 includes an arithmetic circuit 74, an image generation circuit 75, a determination circuit 76, an alert output circuit 77, and a storage circuit 78.

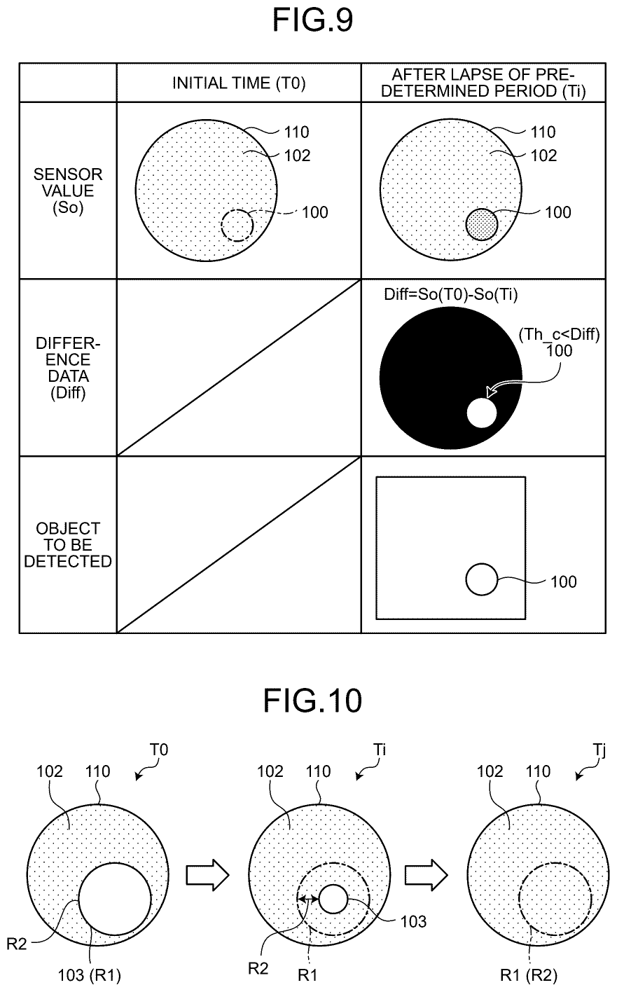

The arithmetic circuit 74 calculates difference data Diff based on baseline data (sensor values So(T0)) and data of the sensor values So(Ti) received from the optical sensor 10. The baseline data is data of the sensor values So obtained by scanning the photodetection elements 30 of the optical sensor 10 in an initial state (for example, at time T0 after power-on). The difference data Diff will be described later with reference to FIGS. 8 and 9.

The image generation circuit 75 generates an image based on the difference data Diff or the data of the sensor values So.

The determination circuit 76 determines the presence or absence of the objects to be detected 100 or the presence or absence of the moisture 103 based on the image data of the image generation circuit 75 and the first threshold Th_c and the second threshold Th_w stored in advance in the storage circuit 78. The determination circuit 76 also determines regions, such as an initial moisture region R1 indicating the presence of the moisture 103 in the initial state, and a moisture evaporation region R2 indicating that the moisture 103 has evaporated, and the positional relation therebetween.

The alert output circuit 77 outputs an alert indicating the detection of the objects to be detected 100 when the objects to be detected 100 are detected. Alternatively, the alert output circuit 77 outputs an alert indicating the positional relation between the objects to be detected 100 and the moisture 103. A display device 86 of the external circuit 85 receives the alert from the alert output circuit 77 and displays the image indicating the detected position of the objects to be detected 100.

The storage circuit 78 stores therein the sensor values So and the baseline data detected by the optical sensor 10, the difference data Diff calculated by the arithmetic circuit 74, and the various preset conditions such as the first threshold Th_c and the second threshold Th_w.

The sensor control circuit 71 illustrated in FIG. 5 is illustrated schematically for ease of understanding. The circuits included in the sensor control circuit 71 are not limited to being configured such that each of the circuits is provided as an individual circuit and may be configured as one integrated circuit (IC).

The following describes a detection method of the detection device 1. As described above, the detection device 1 detects the objects to be detected 100 cultured on the culture medium 102. However, if the moisture 103 adheres to the container 110 or the culture medium 102, the detection device 1 may detect the moisture 103. There is a demand to reduce false detection of the moisture 103 in the detection of the objects to be detected 100 cultured on the culture medium 102. Alternatively, there is a demand to allow the user to visually check the image when the objects to be detected 100 are located so as to overlap the moisture 103.

The following sequentially describes a method for detecting the objects to be detected 100 when the moisture 103 is not present, a method for detecting the moisture 103 when the objects to be detected 100 are not present (or not fully grown), and a method for detecting the objects to be detected 100 when they are located so as to overlap the moisture 103.

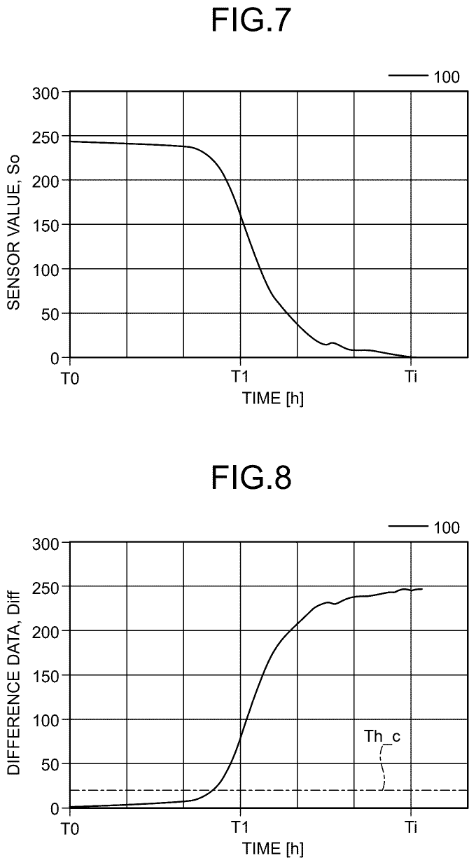

FIG. 6 is a schematic diagram schematically illustrating states of growth of the objects to be detected. FIG. 7 is a graph schematically illustrating a relation between the sensor value and time in the detection of the objects to be detected. FIG. 8 is a graph schematically illustrating a relation between the difference data and time in the detection of the objects to be detected. FIG. 9 is an explanatory chart for explaining the sensor value, the difference data, and the detected objects in the detection of the objects to be detected.

In FIG. 6, the intensity of the light transmitted through the container 110 and the culture medium 102 and received by the optical sensor 10 (hereinafter, referred to as “transmitted light intensity”) is schematically illustrated in different gray shades. That is, the degree of the gray shade illustrated in FIG. 6 corresponds to the magnitude of the sensor values So detected by the optical sensor 10. The shade is illustrated closer to white as the transmitted light intensity (sensor value So) is higher, and illustrated closer to black as the transmitted light intensity (sensor value So) is lower. FIGS. 7 and 8 illustrate the sensor value So and the difference data Diff in the area overlapping the objects to be detected 100 in FIG. 6.

As illustrated in FIGS. 6 and 7, the detection device 1 scans the photodetection elements 30 of the optical sensor 10 at predetermined intervals of time to detect changes over time of the objects to be detected 100 cultured on the culture medium 102. FIG. 6 schematically illustrates the detection results of the objects to be detected 100 at three times T0, T1, and Ti. However, the detection device 1 is not limited to detecting the objects to be detected 100 three times, and repeatedly detects the objects to be detected 100 at the predetermined intervals of time (for example, at intervals of substantially 5 to 10 minutes).

As illustrated in FIG. 6, at time T0 in the initial state, the objects to be detected 100 have not grown, so that the transmitted light intensity across the culture medium 102 in the container 110 is substantially constant. In the following description, the sensor values So at the initial state (time T0) are represented as the baseline data (sensor values So(T0)). At times T1 and Ti when predetermined times have elapsed from time T0, the objects to be detected 100 have grown, and the transmitted light intensity in the area overlapping the objects to be detected 100 gradually decreases. To facilitate understanding of the description, the description will be made assuming that the transmitted light intensity across the culture medium 102 is constant within the container 110 and does not change (or changes at a negligibly small degree) over time.

As illustrated in FIG. 7, the sensor value So exhibits a substantially constant value because the objects to be detected 100 have not grown for a predetermined period of time starting from time T0. As the objects to be detected 100 grow, the sensor value So becomes smaller as the transmitted light intensity decreases.

As illustrated in FIG. 8, the difference data Diff exhibits a tendency of change inverted from that of the sensor value So illustrated in FIG. 7. That is, the difference data Diff exhibits a substantially constant value (Diff=0) during the period in which the objects to be detected 100 have not grown for the predetermined period of time starting from time T0. As the objects to be detected 100 grow, the difference data Diff becomes larger as the sensor value So decreases.

As illustrated in FIGS. 8 and 9, the arithmetic circuit 74 (refer to FIG. 5) calculates the difference data Diff based on the baseline data (sensor values So(T0)) received from the optical sensor 10 and the sensor values So(Ti) at time Ti. The difference data Diff is expressed as Diff=So(T0)−So(Ti). In FIG. 7, the baseline data is the sensor values So at time 0 h (T0).

In FIG. 9, the shade is illustrated closer to black as the difference data Diff is smaller and illustrated closer to white as the difference data Diff is larger. The area illustrated in black in FIG. 9 indicates that the difference data Diff is substantially zero.

As illustrated in FIG. 9, the difference data Diff is larger (illustrated in white) in the area overlapping the objects to be detected 100. The difference data Diff is smaller (illustrated in black) in the area (culture medium 102) not overlapping the objects to be detected 100.

The determination circuit 76 (refer to FIG. 5) compares the difference data Diff with the first threshold Th_c stored in advance in the storage circuit 78. The first threshold Th_c is a reference value indicating that the objects to be detected 100 have been detected. In the area overlapping the objects to be detected 100, the difference data Diff is larger than the first threshold Th_c (Th_c<Diff). In the area not overlapping the objects to be detected 100, the difference data Diff is smaller than the first threshold Th_c (Diff<Th_c). The determination circuit 76 determines that the objects to be detected 100 have been detected when the difference data Diff exceeds the first threshold Th_c.

In the example illustrated in FIGS. 6 to 9, the moisture 103 is not present at time Ti, and an area (initial moisture region R1 to be described later) indicating the presence of the moisture 103 in the initial state (at time T0) is not detected. Therefore, the determination circuit 76 determines that the moisture 103 is not present and the objects to be detected 100 have been detected. The alert output circuit 77 outputs the alert indicating the detection of the objects to be detected 100. The display device 86 of the external circuit 85 displays the image indicating the detected position of the objects to be detected 100 along with the alert indicating the detection of the objects to be detected 100.

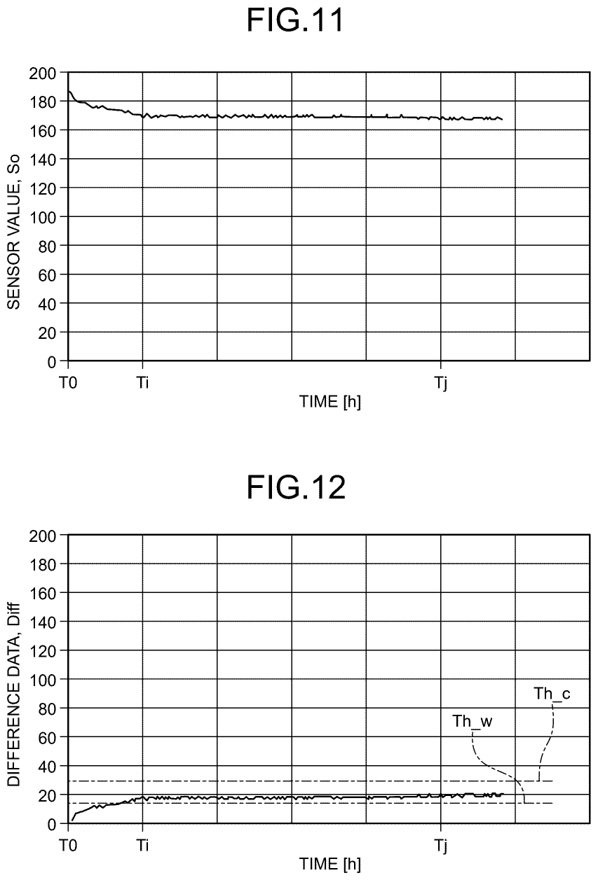

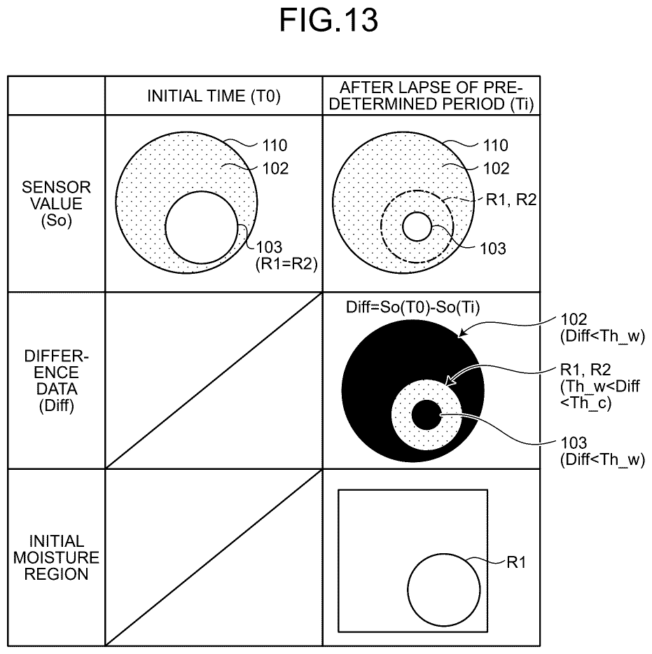

The following describes the method for detecting the moisture 103 when the objects to be detected 100 are not present (or not fully grown). FIG. 10 is a schematic diagram schematically illustrating states of the evaporation of the moisture. FIG. 11 is a graph schematically illustrating a relation between the sensor value and time in the detection of the moisture. FIG. 12 is a graph schematically illustrating a relation between the difference data and time in the detection of the moisture. FIG. 13 is an explanatory chart for explaining the sensor values, the difference data, and the detected initial moisture region in the detection of the moisture.

As illustrated in FIG. 10, at time T0 in the initial state, the transmitted light intensity in the area overlapping the moisture 103 on the container 110 is higher than the transmitted light intensity in an area where the moisture 103 is not present (that is, the culture medium 102). This is due to the following reason. The refractive index n of air is substantially 1. The refractive index n of the container 110 of acrylic is substantially 1.49. The refractive index n of water is substantially 1.33. In an optical path without water (moisture) in the present disclosure, light passes through air (n=1), the container 110 (n=1.49), and then air (n=1) in this order, for example. By contrast, in an optical path through water (moisture), light passes through air (n=1), the container 110 (n=1.49), water (n=1.33), and then air (n=1) in this order, for example. Thus, according to the Fresnel reflectance, the optical path through water (moisture) exhibits higher transmittance than that of the optical path without water (moisture). Herein, the area indicating the presence of the moisture in the initial state (at time T0) is referred to as the initial moisture region R1.

At times T1 and Ti when the predetermined times have elapsed from time T0, the moisture 103 gradually evaporates and the area of the moisture 103 becomes smaller. At time T1, the transmitted light intensity in the area where the moisture 103 has evaporated has decreased and becomes equal to the transmitted light intensity of the culture medium 102. Herein, the area where the moisture 103 has evaporated is referred to as the moisture evaporation region R2. The moisture evaporation region R2 at time T1 is an area between the outer periphery of the moisture 103 and the outer periphery of the initial moisture region R1. As the area of the moisture 103 decreases, the moisture evaporation region R2 becomes larger.

At time Ti, when all the moisture 103 evaporates, the entire initial moisture region R1 becomes the moisture evaporation region R2, and the transmitted light intensity of the initial moisture region R1 becomes equal to the transmitted light intensity of the culture medium 102.

FIG. 11 illustrates the relation between sensor value So and time in the area overlapping the moisture 103 in FIG. 10. As illustrated in FIG. 11, the sensor value So at time T0 exhibits the highest value. As the moisture 103 evaporates over time, the sensor value So becomes smaller as the transmitted light intensity decreases.

As illustrated in FIG. 12, the difference data Diff exhibits a tendency of change inverted from that of the sensor value So illustrated in FIG. 11. That is, the difference data Diff increases as the moisture 103 evaporates from time T0, and after a predetermined period of time has elapsed and the moisture 103 has evaporated, the difference data Diff exhibits a substantially constant value.

As illustrated in FIG. 13, the difference data Diff in the area overlapping the moisture 103 at time Ti is zero (illustrated in black). At time Ti, the area that overlaps the initial moisture region R1 and does not overlap the moisture 103 is the moisture evaporation region R2. The difference data Diff in the moisture evaporation region R2 is larger than that in the moisture 103. The difference data Diff in an area not overlapping the initial moisture region R1 (area outside the initial moisture region R1) is zero (illustrated in black).

More specifically, the determination circuit 76 (refer to FIG. 5) compares the difference data Diff with the first threshold Th_c and the second threshold Th_w stored in advance in the storage circuit 78. The second threshold Th_w is set to a value smaller than the first threshold Th_c, and is a reference value indicating that the moisture 103 has been detected.

The determination circuit 76 extracts a first region (Th_w<Diff) where the difference data Diff is larger than the second threshold Th_w in a distribution of the difference data Diff. The determination circuit 76 determines the first region to be the moisture evaporation region R2. Then, when an area where the difference data Diff is smaller than the second threshold Th_w (Diff<Th_w) is present in an area (including the moisture evaporation region R2 and the moisture 103) inside the outer periphery of the first region, the determination circuit 76 determines the area (Diff<Th_w) to be the moisture 103. When the moisture 103 is detected in the first region (Th_w<Diff), the determination circuit 76 determines an area surrounded by the outer periphery of the first region to be the initial moisture region R1.

As described above, the determination circuit 76 detects the presence of the moisture 103 (Diff<Th_w) within an area where the difference data Diff exceeds the second threshold Th_w and does not exceed the first threshold Th_c (Th_w<Diff<Th_c).

As illustrated in FIGS. 6 to 13, the time-varying changes in the sensor value So and the difference data Diff due to the growth of the object to be detected 100 exhibit the same tendency of change as the time-varying changes in the sensor value So and the difference data Diff due to the evaporation of the moisture 103. Even in this case, the detection device 1 of the present embodiment can determine whether the time-varying changes are due to the moisture 103 or the objects to be detected 100 by comparing the difference data Diff with the first threshold Th_c and the second threshold Th_w in the distribution of the difference data Diff.

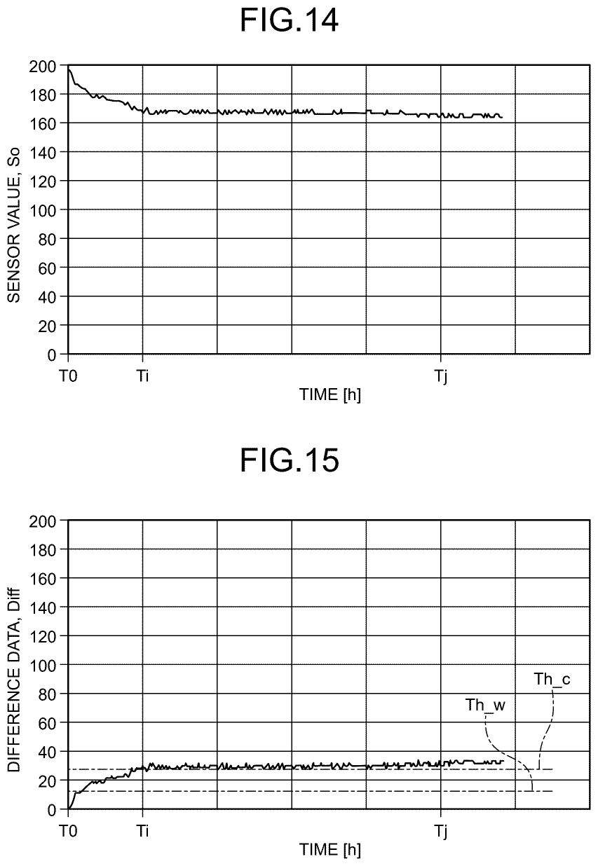

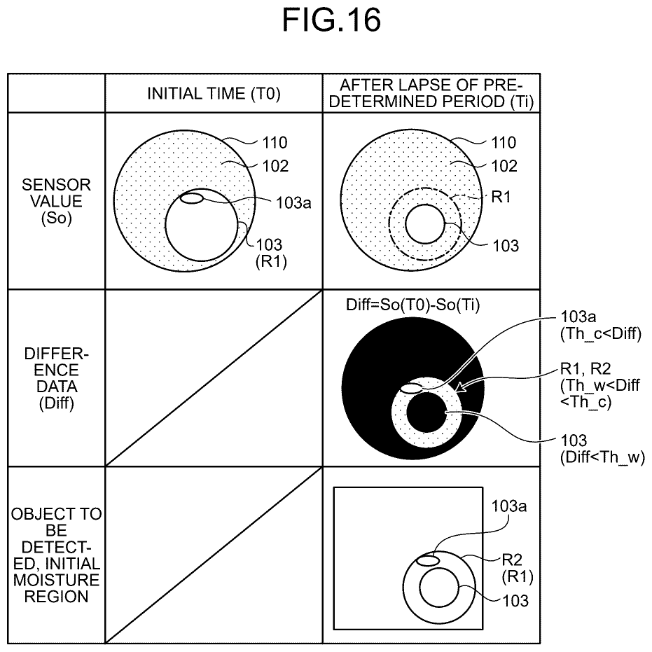

FIG. 14 is a graph schematically illustrating a relation between the sensor value and time in the detection of the moisture according to a modification of the embodiment. FIG. 15 is a graph schematically illustrating a relation between the difference data and time in the detection of the moisture according to the modification. FIG. 16 is an explanatory chart for explaining the sensor values, the difference data, and the detected initial moisture region in the detection of the moisture according to the modification.

FIGS. 14 to 16 illustrate examples of a case where the transmitted light intensity in some region 103a of the moisture 103 is higher at time T0 in the initial state. As illustrated in FIGS. 14 and 15, the change in the sensor value So due to the evaporation of the moisture may increase in the region 103a, causing the difference data Diff to exceed the first threshold Th_c.

As illustrated in FIG. 16, the determination circuit 76 detects the region 103a where the difference data Diff exceeds the first threshold Th_c, in the initial moisture region R1 where the difference data Diff is larger than the second threshold Th_w (Th_w<Diff<Th_c). In this case, it is difficult to determine, by using only the difference data Diff, whether the region 103a is a portion of the moisture 103 or the object to be detected 100. The alert output circuit 77 outputs the alert indicating the detection of the objects to be detected 100 in the initial moisture region R1, based on the determination result from the determination circuit 76. This alert causes the user to view the inside of the container 110 to check that not the objects to be detected 100, but the portion of the moisture 103 has been detected.

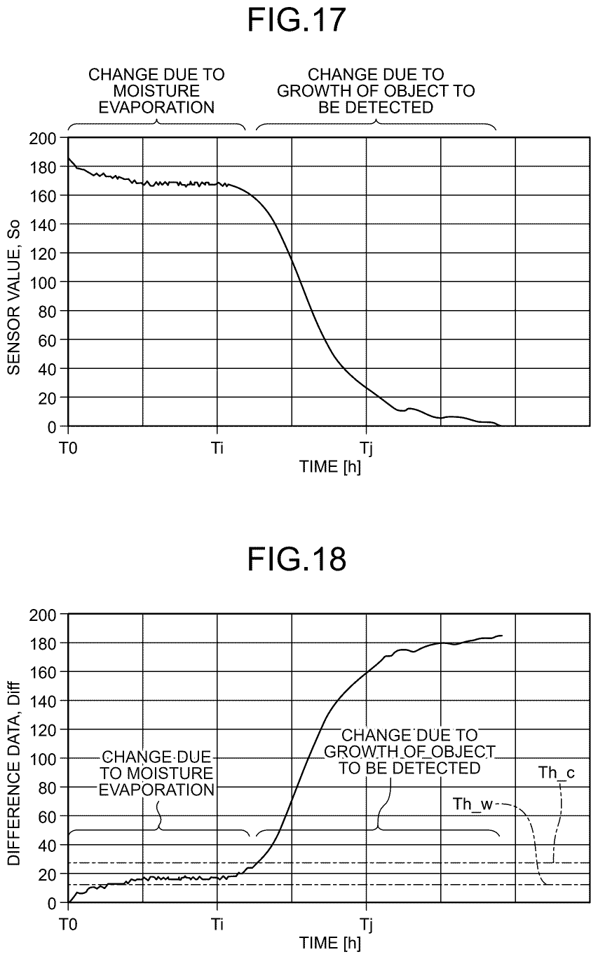

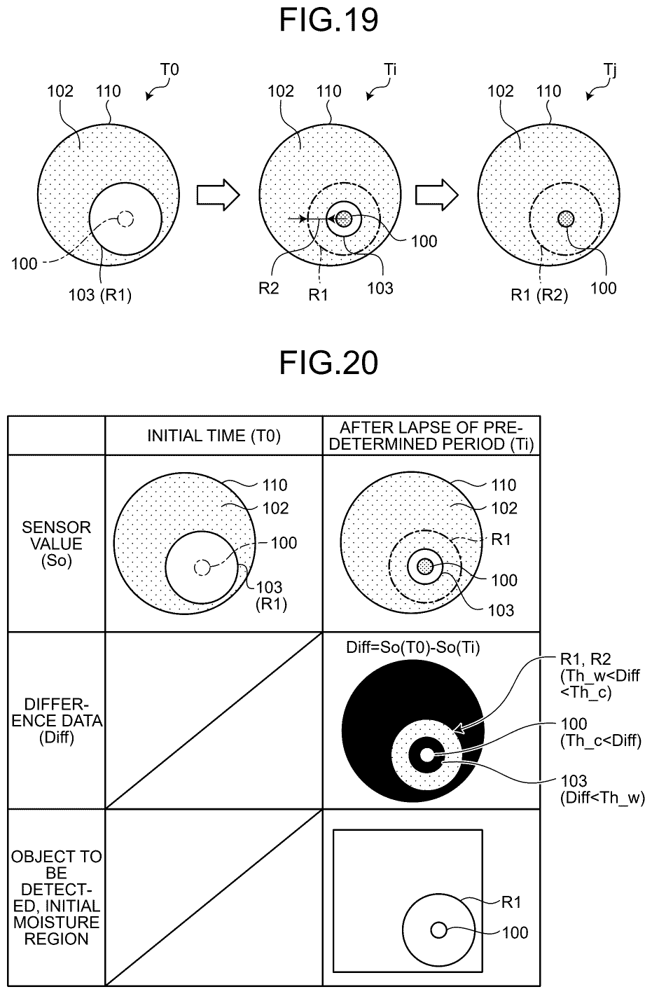

The following describes the method for detecting the objects to be detected 100 when located so as to overlap the moisture 103. FIG. 17 is a graph schematically illustrating a relation between the sensor value and time in the detection of the objects to be detected and the moisture. FIG. 18 is a graph schematically illustrating a relation between the difference data and time in the detection of the objects to be detected and the moisture. FIG. 19 is a schematic diagram schematically illustrating the states of the growth of the objects to be detected and the states of the evaporation of the moisture. FIG. 20 is an explanatory chart for explaining the sensor values, the difference data, and the objects to be detected and the initial moisture region that have been detected in the detection of the objects to be detected and the moisture.

The following describes two examples in each of which the objects to be detected 100 overlap the moisture 103: an example where the objects to be detected 100 grow in the area that overlaps the initial moisture region R1 after the moisture 103 has evaporated; and an example where the objects to be detected 100 grow faster than the evaporation of the moisture 103 (the objects to be detected 100 and the moisture 103 are simultaneously present).

FIGS. 17 and 18 illustrate the example where the objects to be detected 100 grow after the moisture 103 has the evaporated. As illustrated in FIG. 17, the sensor value So exhibits the highest value at time T0. As the moisture 103 evaporates over time, the sensor value So becomes smaller as the transmitted light intensity decreases. As the objects to be detected 100 grow after the moisture 103 has evaporated, the sensor value So becomes smaller as the transmitted light intensity further decreases.

As illustrated in FIG. 18, the evaporation of the moisture 103 makes the difference data Diff larger than the second threshold Th_w and smaller than the first threshold Th_c. After the moisture 103 has evaporated, the growth of the objects to be detected 100 makes the difference data Diff larger than the first threshold Th_c.

Thus, when the objects to be detected 100 grow after the evaporation of the moisture 103, the sensor value So and the difference data Diff exhibit two-step changes. In the changes of the sensor value So and the difference data Diff due to the evaporation of the moisture 103, the same method as that described above with reference to FIGS. 10 to 13 can be applied to determine the initial moisture region R1 and the moisture evaporation region R2. In the changes of the sensor value So and the difference data Diff due to the growth of the objects to be detected 100, the same method as that described above with reference to FIGS. 6 to 9 can be applied to determine the objects to be detected 100.

The determination circuit 76 compares the initial moisture region R1 detected by the evaporation of the moisture 103 with the position of the objects to be detected 100 detected by the growth of the objects to be detected 100. The determination circuit 76 determines that the objects to be detected 100 are present at a position overlapping the initial moisture region R1. The alert output circuit 77 outputs the alert indicating the detection of the objects to be detected 100 in the initial moisture region R1.

FIGS. 19 and 20 explain an example where the objects to be detected 100 grow faster than the evaporation of the moisture 103. As illustrated in FIG. 19, the moisture 103 gradually evaporates over time from time T0. The objects to be detected 100 gradually grow over time from time T0 in the area overlapping the initial moisture region R1. At time Ti before the evaporation of the moisture 103, the objects to be detected 100 and the moisture 103 are simultaneously present, and the objects to be detected 100 overlap the moisture 103. At time Tj after a predetermined period of time has elapsed from time Ti, the moisture 103 has evaporated and the objects to be detected 100 are present in the initial moisture region R1 (moisture evaporation region R2). The following description describes the case where the objects to be detected 100 and the moisture 103 are simultaneously present at time Ti.

As illustrated in FIG. 20, the determination circuit 76 compares the difference data Diff with the first threshold Th_c and the second threshold Th_w. That is, the determination circuit 76 extracts a region in the distribution of the difference data Diff where the difference data Diff is larger than the first threshold Th_c (hereinafter, referred to as “white region”). If the white region (Th_c<Diff) has an area equal to or larger than a predetermined area, the determination circuit 76 determines that the white region (Th_c<Diff) is an object-to-be-detected region indicating the detection of the objects to be detected 100.

Furthermore, in the same way as the example described with reference to FIG. 13, the determination circuit 76 determines the initial moisture region R1, the moisture evaporation region R2, and the moisture 103 by comparing the difference data Diff with the first threshold Th_c and the second threshold Th_w. The method for determining the initial moisture region R1, the moisture evaporation region R2, and the moisture 103 is the same as that described with reference to FIG. 13, and will not be repeatedly described.

The determination circuit 76 compares the initial moisture region R1 with the position of the objects to be detected 100 (white region (Th_c<Diff)) and determines that the objects to be detected 100 overlap the initial moisture region R1. The alert output circuit 77 outputs the alert indicating the detection of the objects to be detected 100 in the initial moisture region R1. This alert allows the user to visually check the inside of the container 110 based on the alert to check whether the detection is detection of the objects to be detected 100 or false detection of the moisture 103.

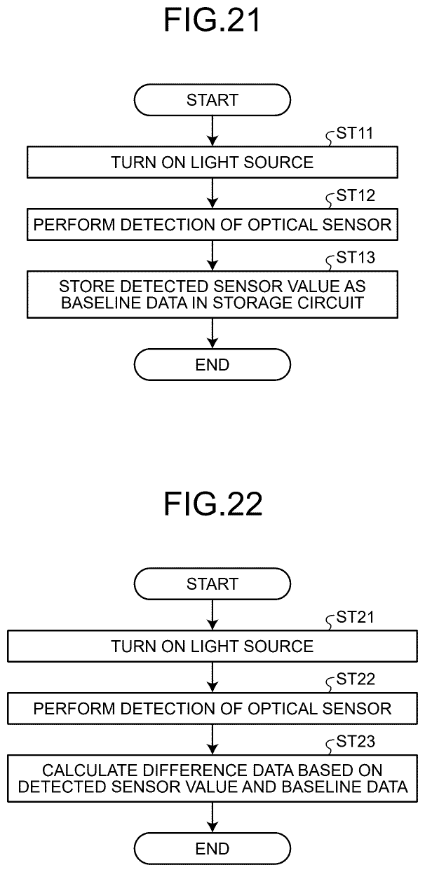

The following describes a detailed method for detecting the objects to be detected 100 and the moisture 103 with reference to FIGS. 21 to 24. FIG. 21 is a flowchart for explaining a method for obtaining the baseline data. As illustrated in FIG. 21, in the initial state after power-on, the control circuit 70 (light source control circuit 72) turns on the light source 80 (Step ST11).

The control circuit 70 (sensor control circuit 71) scans the photodetection elements 30 to perform the detection of the optical sensor 10 (Step ST12). The photodetection elements 30 output the sensor values So each of which corresponds to the light that has been emitted from the light source 80 and transmitted through the container 110 (at least one item of the objects to be detected 100, the culture medium 102, and the moisture 103).

The sensor control circuit 71 acquires the sensor values So in the initial state from the photodetection elements 30 and stores the detected sensor values So as the baseline data in the storage circuit 78 (Step ST13). FIG. 22 is a flowchart for explaining a method for obtaining the difference data. As illustrated in FIG. 22, after a predetermined period of time has elapsed from the initial state (for example, at time Ti), the control circuit 70 (light source control circuit 72) turns on the light source 80 (Step ST21).

The control circuit 70 (sensor control circuit 71) scans the photodetection elements 30 to perform the detection of the optical sensor 10 (Step ST22). The photodetection elements 30 output the sensor values So each of which corresponds to the light transmitted through at least one item of the objects to be detected 100, the culture medium 102, and the moisture 103 at time Ti.

The arithmetic circuit 74 calculates the difference data Diff based on the sensor values So received from the optical sensor 10 and the baseline data received from the storage circuit 78 (Step ST23). The calculation of the difference data Diff is performed for each sensor pixel PX.

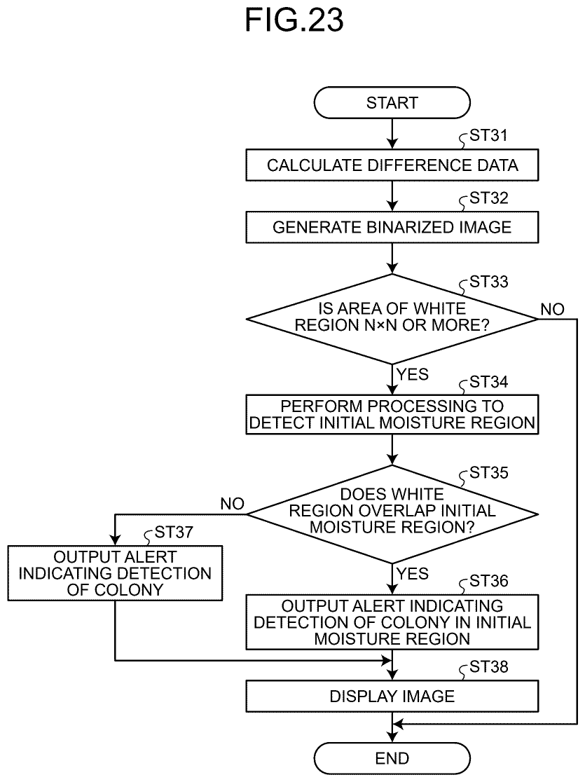

FIG. 23 is a flowchart for explaining a method for detecting the objects to be detected and the output of the alerts. As illustrated in FIG. 23, the arithmetic circuit 74 calculates the difference data Diff at a predetermined timing (for example, time Ti) (Step ST31). The calculation of the difference data Diff at Step ST31 is the same as in the flowchart illustrated in FIG. 22. However, FIG. 23 does not illustrate Steps ST21 and ST22 in the flowchart of FIG. 22.

The image generation circuit 75 generates a binarized image based on the difference data Diff (Step ST32). The image generation circuit 75 compares the difference data Diff with the first threshold Th_c. The image generation circuit 75 sets the sensor pixels PX where the difference data Diff is equal to or larger than the first threshold Th_c (Th_c<Diff) to be the white region (object-to-be-detected region) and sets the sensor pixels PX where the difference data Diff is lower the first threshold Th_c (Diff<Th_c) to be a black region (refer to FIG. 9).

The determination circuit 76 receives the binarized image from the image generation circuit 75 and determines whether the group of the sensor pixels PX in the white region (Th_c≤Diff) is N×N pixels or more (Step ST33). N is the number of pixels indicating that the objects to be detected 100 have grown to be a colony and is set based on past detection data of colonies (objects to be detected 100). The number of pixels N is stored in advance in the storage circuit 78.

If the white region has less than N×N pixels (No at Step ST33), the determination circuit 76 determines that the objects to be detected 100 have not grown, and ends the process of detecting the objects to be detected 100. In this case, the alert output circuit 77 does not output the alert indicating the detection of the colony (objects to be detected 100).

If the white region has N×N pixels or more (Yes at Step ST33), the determination circuit 76 determines that the objects to be detected 100 have grown as the colony and performs processing to detect the initial moisture region R1 (Step ST34).

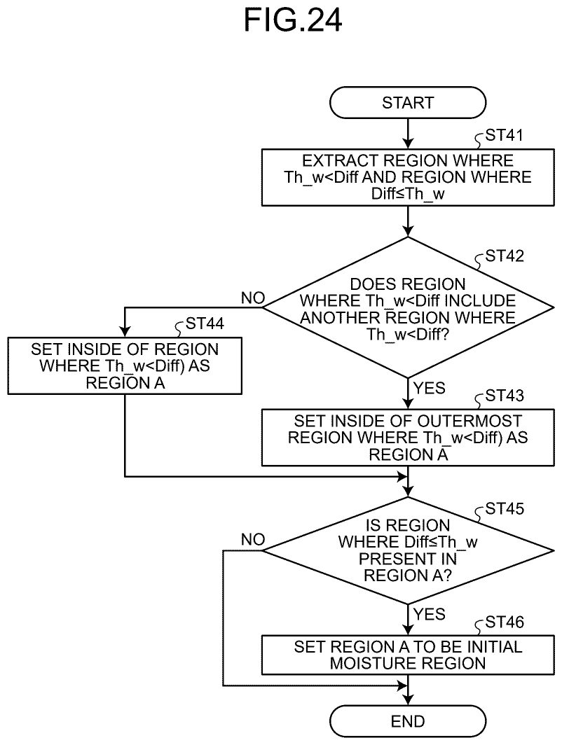

FIG. 24 is a flowchart for explaining a method for detecting the moisture. As illustrated in FIG. 24, the determination circuit 76 compares the difference data Diff obtained at Step ST31 with the second threshold Th_w, and extracts the region where the difference data Diff is larger than the second threshold Th_w (first region: Th_w<Diff) and a region where the difference data Diff is equal to or smaller than the second threshold Th_w (Diff≤Th_w) (Step ST41).

The determination circuit 76 determines whether the region where the difference data Diff is larger than the second threshold Th_w (first region (Th_w<Diff)) includes another region where the difference data Diff is larger than the second threshold Th_w (Th_w<Diff) (Step ST42).

If the other region (Th_w<Diff) is present inside the outermost first region (Th_w<Diff) (Yes at Step ST42), the determination circuit 76 sets the outermost first region (Th_w<Diff) to be a region A (Step ST43). At Step ST43, the case where the other region (Th_w<Diff) is present inside the outermost first region (Th_w<Diff) refers to that a ring-shaped bright-to-dark-to-bright pattern is repeatedly arranged from the outer periphery toward the inner periphery in the distribution of the difference data Diff (refer to FIG. 20).

If the other region (Th_w<Diff) is not present inside the outermost first region (Th_w<Diff) (No at Step ST42), the determination circuit 76 sets the region (Th_w<Diff) as region A (Step ST44). At Step ST44, the case where the other region (Th_w<Diff) is not present inside the outermost first region (Th_w<Diff) refers to that a ring-shaped bright-to-dark pattern is arranged from the outer periphery toward the inner periphery in the distribution of the difference data Diff (refer to FIG.

13), or a case where the region indicating darkness (Diff≤Th_w) is not present inside the first region (Th_w<Diff).

Then, the determination circuit 76 determines whether a second region (Diff≤Th_w) where the difference data Diff is equal to or smaller than the second threshold Th_w is present in region A (Step ST45). If the second region (Diff≤Th_w) is present in region A (Yes at Step ST45), the determination circuit 76 determines region A (that is, the outermost first region (Th_w<Diff)) to be the initial moisture region R1 (Step ST46). The determination circuit 76 determines the second region (Diff≤Th_w) in region A to be an area where the moisture 103 has been detected, and determines an area between the outer periphery of the second region (Diff≤Th_w) and the outer periphery of region A to be the moisture evaporation region R2.

If the second region (Diff≤Th_w) is not present in region A (No at Step ST45), the moisture 103 is determined to be not present, and the detection of the moisture ends. In this case, the determination circuit 76 determines region A to be the moisture evaporation region R2 (=initial moisture region R1).

Referring back to FIG. 23, the determination circuit 76 determines whether the white region (object-to-be-detected region) overlaps the initial moisture region R1 (Step ST35). More specifically, the determination circuit 76 compares the position of the sensor pixel PX indicating the white region obtained at Step ST32 with the initial moisture region R1 obtained at Step ST46.

If the white region (object-to-be-detected region) overlaps the initial moisture region R1 (Yes at step ST35), the determination circuit 76 outputs the determination result to the alert output circuit 77. The alert output circuit 77 outputs the alert indicating the detection of the colony (objects to be detected 100) in the initial moisture region R1, based on the determination result from the determination circuit 76 (Step ST36).

If the white region (objects to be detected 100) does not overlap the initial moisture region R1 (No at Step ST35), the determination circuit 76 outputs the determination result to the alert output circuit 77. That is, the determination circuit 76 determines that the objects to be detected 100 have been detected outside the initial moisture region R1. The alert output circuit 77 outputs the alert indicating the detection of the colony (objects to be detected 100), based on the determination result from the determination circuit 76 (Step ST37).

The display device 86 of the external circuit 85 displays the alert and also displays an image indicating the objects to be detected 100 and the initial moisture region R1 or the image indicating the detected location of the objects to be detected 100 (Step ST38).

As described above, the detection device 1 of the present embodiment compares the difference data Diff with the first threshold Th_c and the second threshold Th_w in the distribution of the difference data Diff to determine, based on the bright/dark pattern of the difference data Diff, whether the objects to be detected 100 have been detected or the objects to be detected 100 in the initial moisture region R1 have been detected.

The processes illustrated in FIGS. 22 and 23 are repeated at predetermined intervals of time and performed a plurality of times from time T0 in the initial state. This operation allows the detection device 1 to observe changes over time concerning the growth of the objects to be detected 100 and the evaporation of the moisture 103. Each of the processes described above can be modified as appropriate. For example, in the process of detecting the objects to be detected 100 illustrated in FIG. 23, the detection of the initial moisture region R1 illustrated at Step ST34 may be performed in advance before the detection process in FIG. 23.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and the modification described above.

What is claimed is:

1. A detection device comprising:
an optical sensor comprising a plurality of photodetection elements arranged in a planar configuration;
a light source configured to emit light to the photodetection elements; and
an object placement member that has a light-transmitting property and on which a plurality of objects to be detected are to be placed, wherein
the light source, the object placement member, and the optical sensor are arranged in the order as listed,
the optical sensor is configured to obtain baseline data in an initial state after power-on,
the detection device is configured to:
obtain difference data based on a difference between the baseline data and sensor values obtained from the photodetection elements at intervals of a predetermined period of time; and
output, when the difference data has exceeded a first threshold, either an alert indicating detection of the objects to be detected in an initial moisture region indicating presence of moisture in the initial state, or an alert indicating detection of the objects to be detected.

2. The detection device according to claim 1, having a second threshold smaller than the first threshold, and configured to:
extract a first region where the difference data is larger than the second threshold in a distribution of the difference data, and
determine, when a second region where the difference data is smaller than the second threshold is present in an area inside an outer periphery of the first region, the first region to be the initial moisture region.

3. The detection device according to claim 2, configured to determine an area between the initial moisture region and the second region to be a moisture evaporation region indicating that the moisture has evaporated from the initial state until a time when the predetermined period of time has elapsed and at which the difference data is obtained.

4. The detection device according to claim 2, configured to determine the second region to be an area where the moisture has been detected at a time when the predetermined period of time has elapsed and at which the difference data is obtained.

5. The detection device according to claim 1, configured to:
extract an object-to-be-detected region where the difference data is larger than the first threshold in a distribution of the difference data, and
output, when the object-to-be-detected region overlaps the initial moisture region, the alert indicating the detection of the objects to be detected in the initial moisture region.

6. The detection device according to claim 1, configured to:
extract an object-to-be-detected region where the difference data is larger than the first threshold in a distribution of the difference data, and
output, when the object-to-be-detected region does not overlap the initial moisture region, the alert indicating the detection of the objects to be detected.

7. The detection device according to claim 1, wherein the photodetection elements are photodiodes.
